(19) United States
(12) Patent Application Publication
(10) Pub. No.: US 2018/0214282 A1
(43) Pub. Date: Aug. 2, 2018

(54) APPARATUS FOR PROCESSING TEETH IN BONE MATERIAL

(71) Applicant: TT TOOTH TRANSFORMER S.R.L., Milan (IT)

(72) Inventor: Elio MINETTI, SPIAZZO (Trento) (IT)

(73) Assignee: TT TOOTH TRANSFORMER S.R.L., Milan (IT)

(21) Appl. No.: 15/747,856

(22) PCT Filed: Jul. 22, 2016
(86) PCT No.: PCT/IB2016/054386

§ 371 Date: Jan. 26, 2018

(30) Foreign Application Priority Data
Jul. 28, 2015 (IT) UB2015A002560

(51) Int. Cl. A61F 2/46; A61C 8/00; A61C 13/00; A61K 6/02

(52) U.S. Cl. CPC A61F 2/4644; A61C 8/0015; A61F 2310/00359; A61K 6/02; A61F 2002/4645; A61C 13/0003

(57) ABSTRACT

Provided is an apparatus for processing teeth in bone material, including a reactor, configured for treating said teeth by immersion in liquids, and including connection means, with a cartridge including a plurality of containers for washing and processing liquids, separated liquid-tight from each other, a reaction chamber configured for containing the teeth during processing, and the washing and processing liquids, commandable transfer means of the liquids from the containers to the reaction chamber configured for placing only one of said containers at a time in fluidic connection with the reaction chamber, ejection means of the liquids from the reaction chamber.

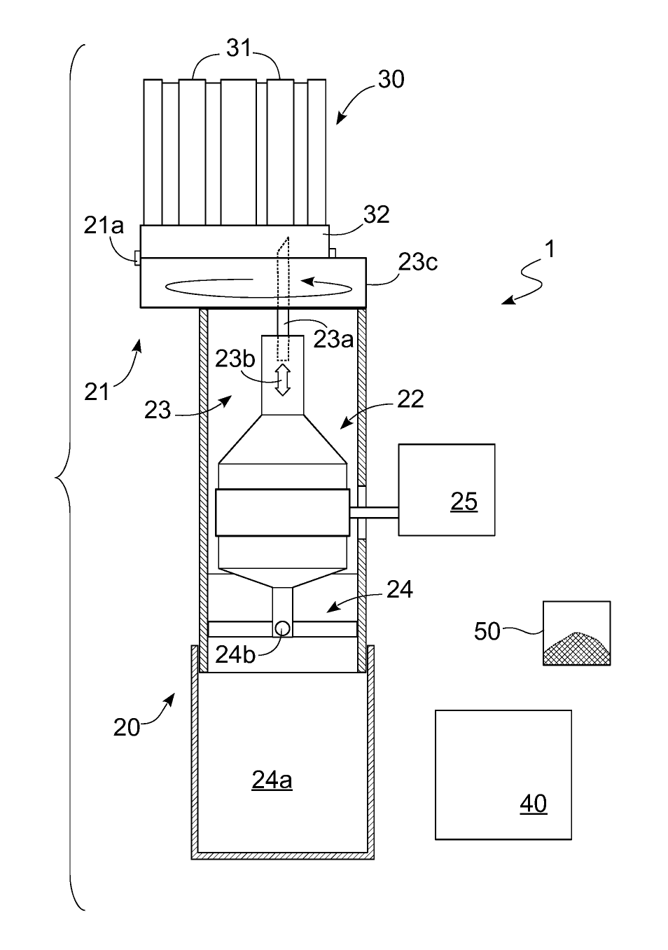

The object of the present invention concerns apparatus for processing teeth in bone material of the type specified in the preamble of the first claim.

Procedures and methods for processing a tooth or portion of tooth in bone material suitable for constituting the base to support a new tooth or for other purposes are currently known.

In particular, the tooth is extracted, washed, broken down and treated with different liquids.

The treatment is carried out, in particular, with mixtures of chloroform, methanol, hydrochloric acid and hydrogen peroxide, for several hours or even days.

The processed tooth is thus a bone powder with the same DNA as the bone of the patient whose tooth was extracted.

The thus formed bone can be used on the same patient as the base for an implant and for the formation and integration of said implant with the bone, for example of the jaw bone, where an artificial tooth is to be fitted.

Said process has been known for about twenty years and is described, for example in the Korean patent applications KR-A-20030068957 and KR-A-19980008980, and also in the scientific article entitled “Development of a novel bone grafting material using autogenous teeth” and published in the journal Oral Surg Oral Med Oral Pathol Oral Radiol Endod 2010; 109:496-503.

Said procedure is extremely advantageous in that it allows greater integration of a prosthesis, in particular of a dental type, on a patient's bones, as well as improved patient recovery.

Apparatus has been produced to realise said process.

However, said apparatus presents important inconveniences.

In fact, said apparatus is extremely complex and costly and furthermore it does not guarantee patient sterility and it does not protect the patient against the risk of cross-infection.

Due to its complexity, it is subject to malfunctioning.

Lastly, said apparatus does not allow a precise and safe dosage and composition of the treatment liquids.

In this situation, the technical task at the base of the present invention is to create an apparatus for processing teeth in bone material, which can substantially remedy the aforesaid inconveniences.

In the sphere of said technical task, it is an important scope of the invention to obtain apparatus for processing teeth in bone material, which is simple, cheap and robust.

Another important purpose of the invention is to create apparatus for processing teeth in bone material that allows a precise and unequivocal supply of treatment liquids.

The technical task and specified purposes are reached in accordance with the claims in the annexed claim 1.

Preferred embodiments are highlighted in the dependent claims.

The characteristics and advantages of the invention are subsequently clarified by the detailed description of preferred embodiments of the invention, with reference to the accompanying drawings, wherein:

The FIGURE shows a diagram of the apparatus for processing teeth in bone material according to the invention.

With reference to the FIGURE, the apparatus for processing teeth in bone material according to the invention is globally indicated with number 1.

It acts in accordance with previously described processes and reactions, in a known manner and is suitable for processing a tooth, preferably a human tooth, in bone material to support a new tooth or for other purposes.

In short, the apparatus 1 comprises a reactor device 20 to realise the chemical reactions, in particular, with liquids, a cartridge 30 for containing the washing and processing liquids, a fragmentation device 40 for teeth, suitable for fragmenting the teeth, a container for fragments 50 of teeth suitable for being placed in the reactor 20.

In detail, the cartridge 30 comprises a plurality of containers 31, for the processing and washing liquids, separated liquid-tight from each other.

The cartridge 30 is preferably cylindrical and the containers 31 are angular sectors of the same cartridge. Preferably, 8 containers are present, at least three of which for processing, in detail, acid solutions, buffer solutions and alcohol, and at least four for washing. The containers 31 are suitable for containing, and preferably, each one contains an adequate and necessary quantity for one single operation.

The cartridge 30 furthermore comprises engaging means with the reactor device 20.

The cartridge 30 furthermore comprises a support element 32, as a base or similar, and means for indicating the position (not illustrated) suitable for allowing the device 20 to read the position of the cartridge 30 and, in particular of the tanks 31.

Finally, the cartridge 30 is preferably of a disposable type.

The reactor device 20 is suitable for treating the teeth by immersion in liquids.

Furthermore, in short, it comprises connection means 21 with the cartridge 30, suitable for putting the cartridge 30 in mechanical connection with the reactor device 20, a reaction chamber 22, suitable for containing the teeth during processing, and the processing and washing liquids, commendable transfer means 23 of the liquids from the containers 31 to the reaction chamber 22 and ejection means 24 of the liquids in the reaction chamber 22.

In greater detail, the connection means 21 comprise a mechanical support 21a, of a known type, such as a container or the like, for the cartridge 30, which is preferably suitable for blocking the cartridge 30 in a vertical direction.

The reaction chamber 22 is suitable for containing the teeth and liquids during the reactions and washes. It is preferably made of metal, more preferably in stainless steel, and is suitable for being sterilised in autoclave or disposable. The reaction chamber 22 preferably has a cylindrical shape and a central portion with a greater diameter, where said reactions take place. Furthermore, it may comprise openings for loading the teeth or for inspections.

Level with the reaction chamber 22, heating and vibrating means 25 are also present for the reaction chamber 22 and the liquids contained, preferably of an electrical type and preferably elastically connectable and separable from the reaction chamber 22.

The commendable transfer means 23 are suitable for placing one single container 31 at a time in fluidic connection with the reaction chamber 22.

Furthermore, they are opportunely suitable for transferring the liquid from the containers 31 to the reaction chamber 22 by gravity. Furthermore, advantageously, the transfer means 23 are suitable for transferring all of the liquid contained in a said container 31 continuously and do not comprise check valves for the flow of fluid. All of the contents of the containers 31 can and must be poured into the reaction chamber 22 in one single operation, since the liquid is already correctly dosed and made up in the cartridge. The reactor device 20 therefore has a simple and compact structure.

Preferably, the transfer means 23 consist of a hollow needle 23a and first handling means 23b of said needle 23a. The needle 23a, is therefore suitable for perforating one of the containers set vertically above the same needle 23a.

The transfer means 23 furthermore comprise second handling means 23c of the cartridge 30, suitable for positioning a specific tank above the needle 23a and consequently allowing the connection between the same hollow needle 23a and a selected tank 31. The second handling means 21c preferably comprise a rotary engine, opportunely electric, more preferably a stepper motor. Lastly, the connection means 21 comprise means for reading (not illustrated) the position of the cartridge 31, interacting with the means for indicating the position of the cartridge 30 that are suitable for allowing the device 20 to read the position of the cartridge 30 and in particular of the tanks 31 in relation to the device 20 and in particular to the needle 23a.

The ejection means 24 preferably comprise a drainage tank 24a, suitable for containing the liquids after the treatment that are preferably joined in a releasable manner to the rest of the device 20. The drainage tank 24a and the reaction chamber 22 are preferably jointly connected for the passage of fluid and separated by a discharge valve 24b, which can be activated electrically.

Preferably, the transfer of fluids from the reaction chamber 22 to the drainage tank 24a takes place by gravity.

The fragmentation device 40 of the tooth is suitable for fragmenting said tooth, or a portion of tooth, into particles of between 0.3 mm and 0.8 mm in diameter. It comprises a closed container and an electric mill, housed in said container.

Finally, the container for fragments 50 of teeth is suitable for being placed inside the reaction chamber 22, which is suitable for keeping said fragments of teeth inside it and permeable to the liquids during the reactions, so the fragments of tooth come into contact with the washing and reaction liquids.

The working of the apparatus 1 for processing teeth in bone material, previously described in structural terms, is as follows.

A tooth is extracted from the patient.

The same tooth is put into the container of the fragmentation device 40 where it is broken down by the electric mill into particles of between 0.3 mm and 0.8 mm in diameter.

The fragments are placed in the container 50, which is, in turn, in the reaction chamber 22 of the device 20.

At the same time, a cartridge 30 is positioned level with the connection means 21.

The reactor apparatus 20 is then started.

The means for reading the position of the cartridge 31, part of the device 20, read the position of the containers 31 of the cartridge 30, and the second handling means 23c of the cartridge 30, position a special tank above the needle 23a. The first handling means 23b move the hollow needle 23, which perforates the selected container 31.

The liquids contained in the selected container 31 fall into the reaction chamber 22 and come into contact with the container 50, triggering a special reaction or wash with the fragments of tooth.

When the treatment, which is carried out in a set period of time, is finished, the drainage valve 24b is opened with the special liquid and the liquid is transferred to the drainage tank 24a.

Then the cartridge 30 can be moved again, by the second handling means 23c, and a new container can be 30 perforated and its contents transferred to the reaction chamber 22 and subsequently to the drainage tank 24a.

Preferably, wash cycles are alternated with washing and reaction liquids, preferably carried out with HCl, H₂O₂ and alcohol, with times of a known type.

When said operations are finished, the container 50 is extracted and the fragments of teeth, processed into bone fragments, are extracted and utilised.

The cartridge 30, which is now empty, is thrown away and at least portions of the reaction device 20, as well as the container for fragments 50 and the fragmentation device 40 are sterilized in autoclave. Alternatively, the container for fragments 50 is of a disposable type.

The apparatus for processing teeth in bone material according to the invention achieves important advantages.

In fact, the apparatus 1 is very simple, cheap and robust.

Furthermore, the use of the pre-loaded disposable cartridge 30 allows precise dosing and correct composition of the washing and reaction liquids.

The invention is susceptible to variations, which fall within the scope of the inventive concept, defined by the claims.

In this sphere, all of the details can be replaced by equivalent components and any materials, shapes and sizes can be used.

1. The apparatus for processing teeth in bone material comprising a reactor device, configured to treat said teeth by immersion in liquids,
said reactor device comprising:
connection means to a cartridge, comprising a plurality of containers for said processing and washing liquids, separated liquid-tight from each other,
a reaction chamber configured to contain said teeth during said processing and said processing and washing liquids,
commandable transfer means of said liquids from said containers to said reaction chamber configured to place in fluidic connection only one at a time of said containers with said reaction chamber, and
ejection means of said liquids from said reaction chamber.

2. The apparatus according to claim 1, wherein said transfer means are configured to transfer by gravity said liquid from said containers to said reaction chamber.

3. The apparatus according to claim 1, wherein said transfer means are configured to transfer all the liquid contained in a said container continuously and do not comprise check valves of said fluid.

4. The apparatus according to claim 1, wherein said transfer means comprise handling means of said cartridge, so as to place a selected container in a specific position configured to allow said arrangement in a fluidic through connection of said container with said reaction chamber.

5. The apparatus according to claim 1, wherein said ejection means comprise a drainage tank.

6. The apparatus according to claim 5, wherein said ejection means are configured to transfer by gravity said liquid from said reaction chamber to said drainage tank.

7. The apparatus according to claim 1, wherein said reactor device comprises heating and vibrating means for said reaction chamber and the liquids contained therein.

8. The apparatus according to claim 1, comprising said cartridge, wherein said cartridge (30) is a disposable cartridge.

9. The apparatus according to claim 1, comprising a fragmentation device of a tooth, configured to fragment a tooth, or a portion of tooth, into particles of between 0.3 mm and 0.8 mm in diameter.

10. The apparatus according to claim 1, comprising a container for fragments of teeth, configured to be placed inside said reaction chamber, configured to keep said tooth fragments inside it during the reactions, and permeable to liquids.
